(19) United States
(12) Patent Application Publication
(10) Pub. No.: US 2008/0161821 A1
(43) Pub. Date: Jul. 3, 2008

(54) Surgical Tool for Insertion of Spinal Prosthesis

(75) Inventor: Eric S. Heinz, Memphis, TN (US)

Correspondence Address: COATS & BENNETT/MEDTRONICS, 1400 CRESCENT GREEN, SUITE 300, CARY, NC, 27518, US

(73) Assignee: Warsaw Orthopedic, Inc., Warsaw, IN (US)

(21) Appl. No.: 11/549,661

(22) Filed: Oct. 16, 2006

(51) Int. Cl. A61F 2/00

(52) U.S. Cl. 606/99

(57) ABSTRACT

A surgical tool for manipulating a surgical insert selectively clamps the insert between one or more moveable arms and a distal tip portion of a driver shaft assembly. The insert may be clamped or released depending on the position of a slider associated with the driver tip portion, and the slider may also function to help auto-release the insert. In a forward clamping position, the slider urges the arm(s) toward the driver tip portion to clamp the insert therebetween. In a rearward release position, the slider allows the arm(s) to move away from the driver tip portion to release the insert. The tool may also or alternatively allow for the tool's handle to be either longitudinally coupled or longitudinally decoupled from the driver shaft assembly. When decoupled, the driver shaft assembly may be impacted to help drive the insert into the desired location.

### BACKGROUND

The invention relates to a tool and method for inserting prostheses, such as artificial discs and cages, within an intervertebral space during a surgical procedure.

Spinal surgery involves many challenges, with the long-term health and mobility of the patient often depending on the surgeon's technique and precision. One type of spinal surgery involves the removal of the natural disc tissue that is located between adjacent vertebral bodies. Procedures are known in which the natural, damaged disc tissue is replaced with an interbody cage or fusion device, or with a disc prosthesis. During the insertion of an artificial disc prosthesis, one challenge is positioning the implant properly in the space between adjacent bony portions. Insertion can be difficult or time consuming if the bony portions are spaced too close together, or if the adjacent tissue, nerves or vasculature impedes access to or placement of the implant. Improper placement of the prosthesis can lead to pain, postural problems, and/or impede freedom of movement. While a number of specialized tools have been developed to facilitate the placement of such prostheses, there remains a need for alternative tools.

### SUMMARY

In one illustrative embodiment, a surgical tool for manipulating a surgical insert selectively clamps the insert between one or more moveable arms and a distal tip portion of a driver shaft assembly. The insert may be clamped or released depending on the position of a slider associated with the driver tip portion. In a forward clamping position, the slider urges the arm(s) toward the driver tip portion to clamp the insert therebetween. In a rearward release position, the slider allows the arm(s) to move away from the driver tip portion to release the insert. The slider may also function to help auto-release the insert from the tool. One such embodiment comprises: a housing; an elongate driver moveably coupled to the housing and forwardly extending therefrom, the driver having a distal tip portion; a first forwardly extending arm coupled to the driver tip portion and having a first distal end portion biased away therefrom; a slider slidably coupled to the driver tip portion and moveable between a forward clamping position and a rearward release position; wherein moving the slider from the release position to the clamping position urges the distal end portion of the first arm toward the driver tip portion for gripping the surgical insert therebetween; and wherein moving the slider from the clamping position to the release position allows the distal end portion of the first arm to move away from the driver tip portion for releasing the surgical insert.

In another embodiment, a surgical tool for manipulating a surgical insert allows for the handle of the tool to be either longitudinally coupled or longitudinally decoupled from the driver shaft assembly. When decoupled from the handle, the driver shaft assembly may be impacted, such as by a mallet, to help drive the insert into the desired location. One such embodiment comprises: a housing having a bore therethrough with first threads; an elongate driver moveably coupled to the housing and extending through the bore to forwardly extend from the housing; wherein the driver comprises: a distal tip portion for mating with the insert; a proximal drive force-receiving end section disposed generally opposite the tip portion; an intermediate threaded section disposed between the force-receiving end section and the tip portion and having second threads configured to engage the first threads; a bridging section disposed between the intermediate section and the force-receiving end section; the bridging section smaller than the intermediate section; wherein, when the first threads engage the second threads, the driver couples to the handle for longitudinal movement therewith; wherein, when the first threads are aligned with the bridging section, the driver is longitudinally decoupled from, and slidable relative to, the handle.

Various other aspects and embodiments are also disclosed, which may be used alone or in any combination. In addition, various methods of using the various surgical tool embodiments for manipulating a surgical insert are disclosed.

### BRIEF DESCRIPTION OF THE DRAWINGS

FIG. 1 shows a surgical insert suitable for use with the surgical tool.

FIG. 2 shows a perspective view of a surgical tool according to one embodiment with the insert coupled thereto.

FIG. 3 shows one embodiment of a handle.

FIG. 4 shows one embodiment of a driver shaft assembly in side view.

FIG. 5 shows a perspective view of a driver tip portion of the driver shaft assembly of FIG. 4.

FIG. 6 shows an underside perspective view of a clamping arm according to one embodiment.

FIG. 7 shows a perspective view of a slider according to one embodiment.

FIG. 8 shows a side view of a portion of the surgical tool of FIG. 2 in the clamping position.

FIG. 8A shows a top view of the surgical tool of FIG. 8 with insert removed for clarity.

FIG. 9 shows a partial sectional view along line IX-IX of FIG. 8A.

FIG. 10 shows a side view of a portion of the surgical tool of FIG. 2 in the release position.

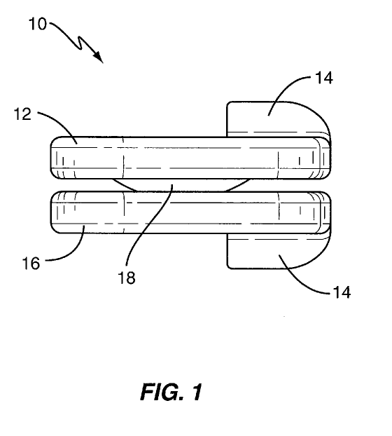

### DETAILED DESCRIPTION

Illustrative embodiments of the present invention include a surgical tool 20 and/or a method of using a surgical tool 20, typically in association with a medical insert 10. One common example of such a medical insert 10 is a disc prosthesis. As such, the discussion below uses a disc prosthesis as an illustrative example of a surgical insert 10. The particular disc prosthesis used for illustrative purposes includes a superior plate 12, an inferior plate 16, and a nucleus 18. The superior plate 12 and the inferior plate 16 are moveable with respect to each other, and the nucleus 18 is generally captured between the two plates 16,18. For further information on the disc prosthesis 10, attention is directed to U.S. patent application Ser. No. 11/343,954, filed 31 Jan. 2006, which is incorporated herein by reference in its entirety. However, it should be understood that the present invention is not limited to use with the particular disc prosthesis shown, and may instead be used with any suitable insert 10.

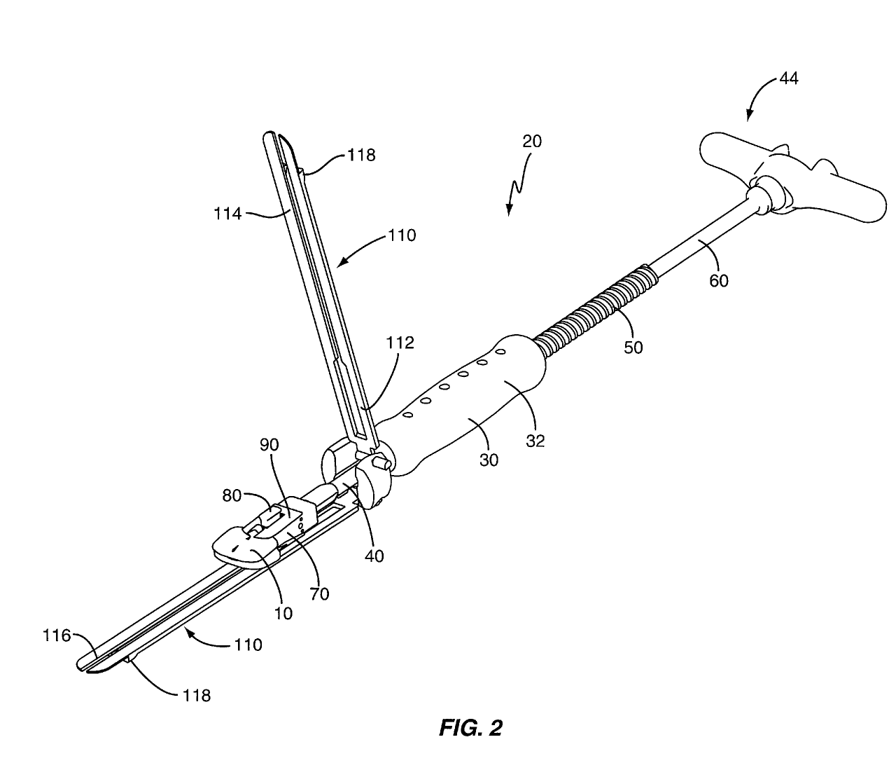

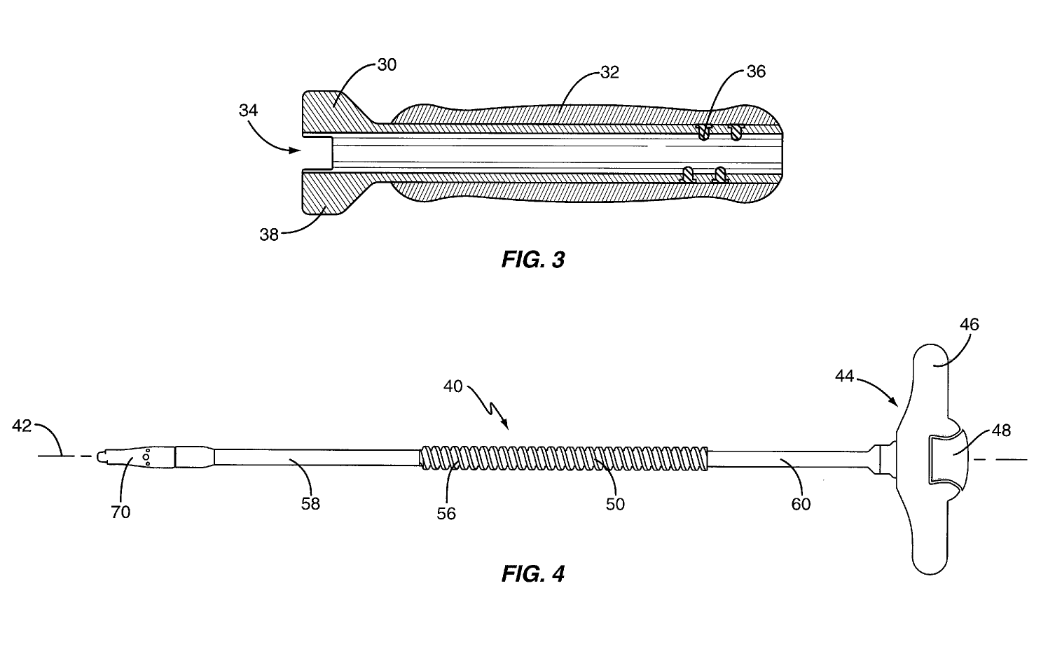

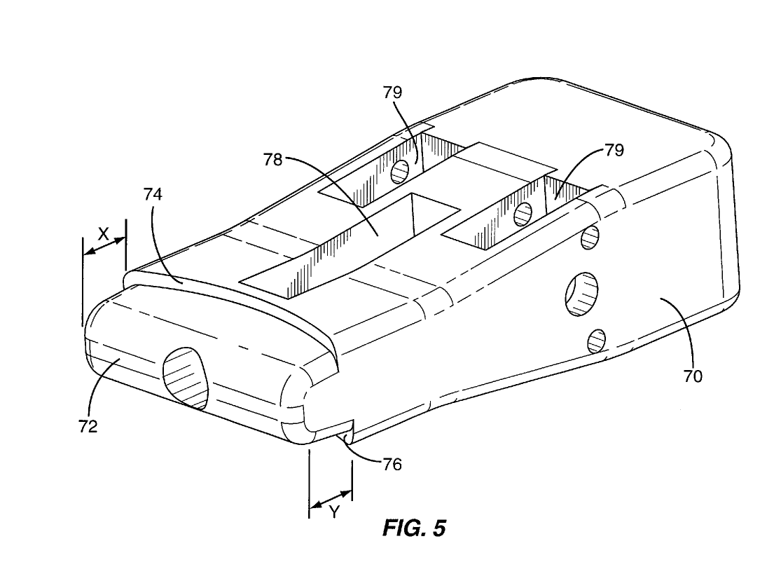

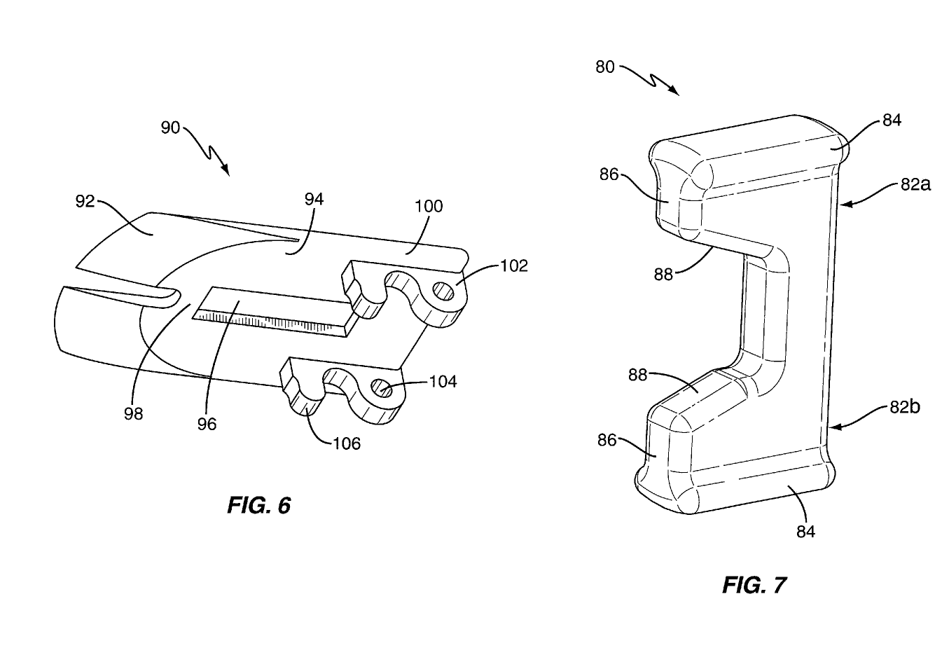

The tool 20 according to one embodiment is shown in FIG. 2. The tool 20 generally includes a handle 30, a central driver shaft assembly 40, a pair of moveable clamping arms 90, and a slider 80. In general terms, the driver shaft assembly 40 is moveable relative to the handle 30 during the initial installation process. The insert 10 may be attached to the driver shaft assembly 40 or released therefrom. To achieve this in some embodiments, the clamping arms 90 may be positioned relatively close to the tip portion 70 of the driver shaft assembly 40 to clamp the insert therebetween, or may be positioned relatively away from the tip portion 70 to release the insert 10. The slider 80 helps control the position of the clamping arms 90, thereby controlling whether the insert is attached to the tool 20 or released. As the insert 10 is installed, the slider 80 abuts against the nearby bone and moves rearward, thereby allowing the clamping arms 90 to release the insert 10. Thus, the illustrated embodiment of the tool 20 grips the insert 10 against the tip portion 70 of the driver shaft assembly 40, and self-releases the insert 10 when appropriate. In addition, some embodiments have a novel design of the driver shaft assembly 40 that allows the driver shaft assembly to be longitudinally decoupled from the handle to allow for impact loads to be applied to the driver shaft assembly while allowing the handle to remain stationary during a portion of the insert installation process.

The illustrated handle 30 is a generally elongate hollow body used to grip and stabilize the tool 20. The handle 30 includes an inner bore 34 for accepting the driver shaft assembly 40. The inner bore 34 advantageously includes threads 36 for mating with corresponding threads 56 on the driver shaft assembly 40; this allows driver shaft assembly 40 to be advanced by a conventional screwing action while the surgeon holds handle 30. These threads 36 may be formed as a conventional single continuous thread, as multiple threads, discontinuous threads, or any other like structure. In one embodiment, the threads 36 are formed by inwardly extending spherical tipped pins that are affixed to the handle 30. The outer portion of the handle 30 may, if desired, include a suitable grip 32, such as an elastomeric grip. Further, the handle 30 may advantageously also include suitable mounting flange(s) 38 for mounting optional distraction blades 110, see the discussion below.

The driver shaft assembly 40 is an elongate body or collection of bodies used to move the insert 10 during installation. The driver shaft assembly 40 extends in a longitudinal proximal-distal direction, generally along axis 42, and is advantageously generally straight. The illustrated driver shaft assembly 40 may be thought of as having several sections or regions: a proximal portion 44, a bridging portion 60, a drive portion 50, an extension section 58, and a distal tip portion 70. The proximal portion 44 includes a pair of outwardly extending flanges 46 used by the surgeon to apply a rotational force. These flanges 46 may be covered with a suitable covering, such as silicone, if desired. The proximal endface of this section may include an impact block 48 that is advantageously aligned with axis 42, such as in a suitable recess formed between flanges 46. The impact block 48 is, as described further below, intended to receive an impact force (e.g., hammer strike) that is used during the later portion of the installation process to seat the insert 10 in the corresponding bone segment(s). As such, the impact block 48 should be made of a suitable sturdy material, such as stainless steel or other surgical grade metallic material. If desired, the impact block 48 may be removable from the remainder of the driver shaft assembly 40; or, if desired, the impact block 48 may be covered by the covering on the flanges 46 so as to be effectively embedded therein. Because the proximal portion 44 initially receives the impact and rotational forces, this section may be referred to as the drive-force-receiving end section 44.

The bridging portion 60 extends forwardly from the proximal section 44, and connects the proximal section 44 to the drive portion 50. The bridging portion 60 typically takes the form a simple cylindrical shaft with a relatively smooth outer surface of reduced diameter. The bridging portion 60 is advantageously smaller in size (e.g., diameter) than the inner thread diameter of the drive portion's threads 56. Depending on the relative position of the handle 30 and the driver shaft assembly 40, the bridging portion 60 may be disposed in the inner bore 34 of the handle 30. Thus, for some longitudinal positions of the driver shaft assembly 40, the bridging portion 60 is aligned with the handle's inner threads 36. With such an alignment, the driver shaft assembly 40 becomes longitudinally slidable relative to the handle 30, rather than longitudinally coupled thereto. This feature may be advantageously used during the final portions of the insert installation process, as discussed further below.

The drive portion 50 is intended to co-operate with the handle 30 to control the longitudinal motion of the driver 40 during the initial portions of the installation process. The drive portion 50 includes an external thread 56 that is engagable with the handle's internal threads 36. Threads 56 are advantageously formed as a single continuous thread, but, like threads 36, may alternatively be formed as multiple threads, discontinuous threads, or any other like structure. When threads 56 engage threads 36, the driver 40 is longitudinally coupled to the handle 30 so as to move therewith. Of course, rotation of the drive section 50 in a first direction advances the driver shaft assembly 40 relative to the handle 30, while rotation in the opposite direction retracts the driver shaft assembly 40.

The extension section 58 extends forwardly from the drive section 50 and typically takes the form of a simple cylindrical shaft. The distal end of the extension section 58 may include a suitable bore or other structure (not shown) for rotatably supporting the distal tip portion 70.

The distal tip portion 70 extends forwardly from the extension section 58 and is advantageously rotatably coupled thereto. The illustrated tip portion 70 includes a distal endface 72 and a pair of abutment faces 74,76 proximally spaced from the endface 72. The abutment faces 74,76 may be equally or unequally spaced from endface 72. For example, the upper abutment face 74 may be spaced distance X from endface 72 and the lower abutment face 76 may be spaced a different distance Y. Rearward from the abutment faces 74,76, the illustrated tip portion 70 includes a through slot 78 for receiving slider 80. This slot 78 is advantageously centrally disposed on tip portion 70, so as to be aligned with axis 42. The illustrated tip portion 70 further includes an upper and lower pair of apertures 79 for receiving the clamping arms 90. These apertures 79 are advantageously disposed laterally on either side of, and slightly rearward of, slot 78. Internally, the illustrated tip portion 70 includes a suitable chamber or chambers for mounting bias elements 126,124 for the slider 80 and the clamping arms 90, respectively. Additionally, while not required, the lateral width of the tip portion 70 is advantageously narrower (or at least not wider) than the width of the narrowest insert 10 intended to be used therewith, so as to not present additional insertion challenges.

The clamping arms 90 are mounted to the tip portion 70 of the driver shaft assembly 40 and typically take the form of forwardly extending plate-like structures. The clamping arms 90 are advantageously substantially identical, although this is not required. The illustrated clamping arm includes a forward gripping area 92, and intermediate area 94 and a rearward mounting area 100. The intermediate area 94 is advantageously generally flat with a slot 96 therethrough for receiving the slider 80. The forward portion of the slot 96 is advantageously closed by a laterally running cross piece 98. The gripping area 92 extends forwardly from the intermediate area 94, and advantageously tapers with respect thereto. The inner face of the gripping area 92 may, if desired, be suitably configured to enhance the gripping action of the clamping arms 90. For example, the inner face may have a grip-enhancing coating, may be etched, may be textured, or may otherwise be treated and/or formed to have enhanced gripping. The gripping area 92 may be continuous or may be split into multiple portions, as is desired; however, the gripping area 92 advantageously extends laterally outward a significant degree on both sides of slot 96. The mounting area 100 may include a pair of inwardly extending mounting flanges 102. Each of the mounting flanges 102 may include a suitable hole 104 for a pivot pin, and a protrusion 106 distally located relative to hole 104. The illustrated clamping arms 90 are pivotally joined to the tip portion 70 of the driver shaft assembly 40 by inserting mounting flanges 102 into the corresponding apertures 79, and inserting a suitable pivot pin 105 through the sidewall of the driver tip portion 70 and holes 104. When mounted, the clamping arms 90 extend forwardly so that the gripping area 92 is proximate the abutment faces 74,76 and/or endface 72 of the driver tip portion 70.

The gripping area 92 of the clamping arms 90 are biased away from the driver tip portion 70. For example, a biasing element 124 associated with driver tip portion 70 may urge each clamping arm 90 outward. One or more torsion springs may be used as the biasing element 124, although this is not required, and the spring(s) 124 may be disposed in driver tip portion 70 and bear against mounting flange protrusions 106. Further, it is envisioned that some embodiments of the clamping arms 90 will be relatively rigid, so as to maintain their generally plate-like form during use. As such, the biasing force of the torsion spring 124 will cause the clamping arms 90 to be urged to pivot outward about pivot pins 105, thereby biasing the gripping areas 92 away from the driver tip portion 70.

The slider 80 may take the form of a generally I-shaped cross-section body with upper and lower portions 82a,82b, as shown in FIG. 7. The upper portion 82a and lower portion 82b are advantageously substantially mirror images of each other, and may be snap-fit or otherwise jointed together during assembly. The upper and lower portions 82a,82b each include a retention flange 84, an abutment face 86, and an engagement face 88. Using the upper portion 82a as an example, the retention flange 84 extends laterally outward along the length of the slider body and forms an uppermost extent of the upper portion 82a. The retention flange 84 is advantageously laterally wider than driver tip portion slot 78 so as to help retain slider 80 in slot 78. In addition, retention flanges 84 aid in guiding the slider 80 as it moves, as discussed further below. The abutment face 86 is forward facing and is advantageously generally vertical. The engagement face 88 extends rearward from the inward end of the abutment face 86, toward the centerline of slider 80. The engagement face 88 of upper portion 82a is sloped downward, while the engagement face 88 of lower portion 82b is sloped upward. These engagement faces 88 may advantageously be relatively straight, but this is not required, and monotonically curving or other shapes may alternatively be used.

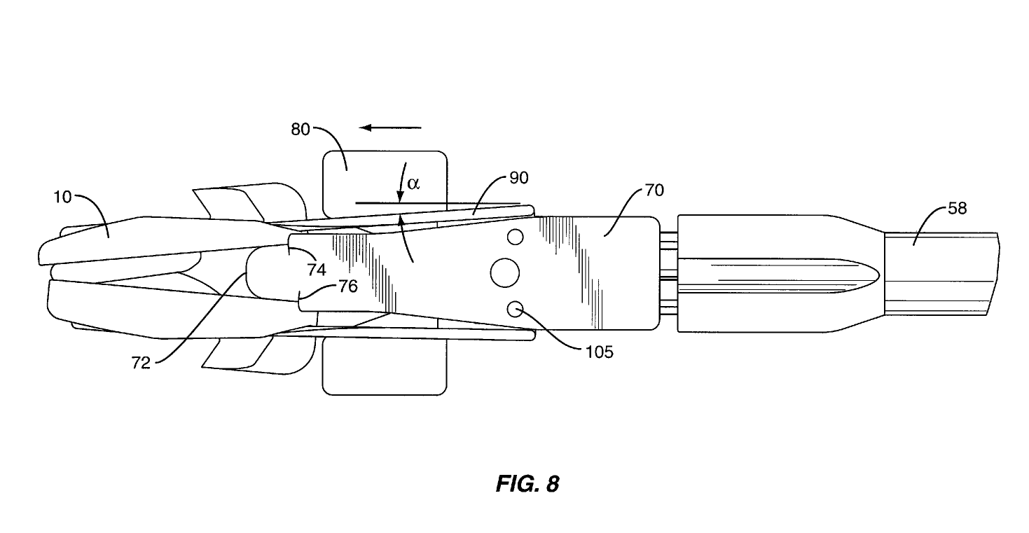

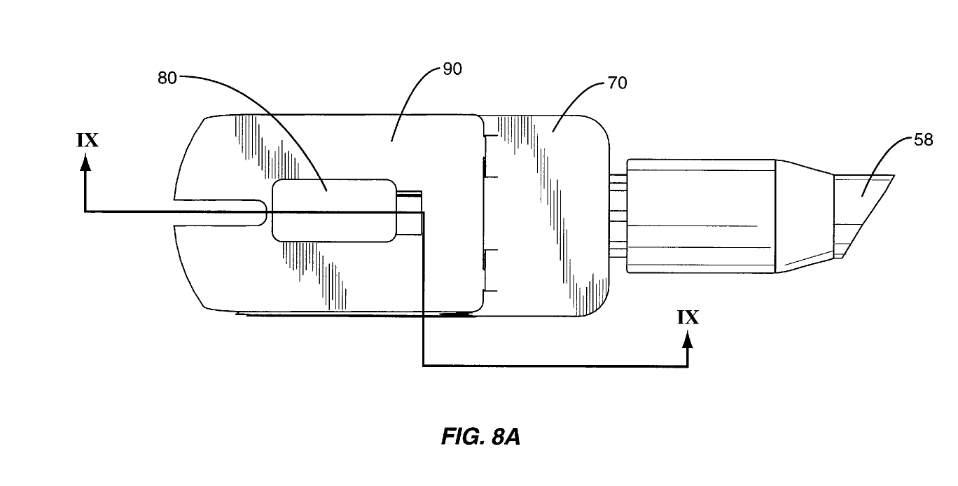

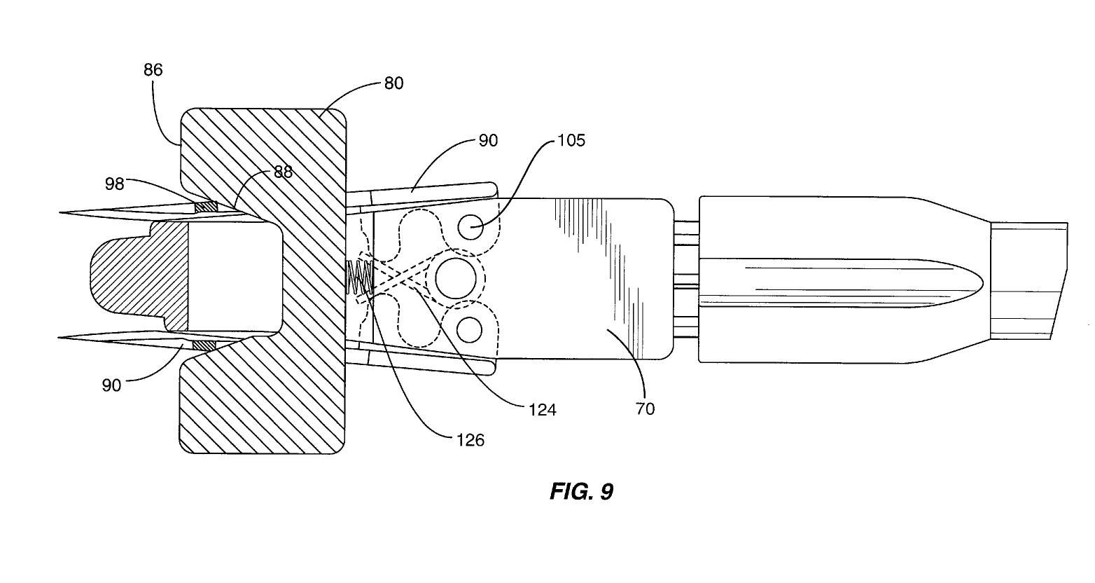

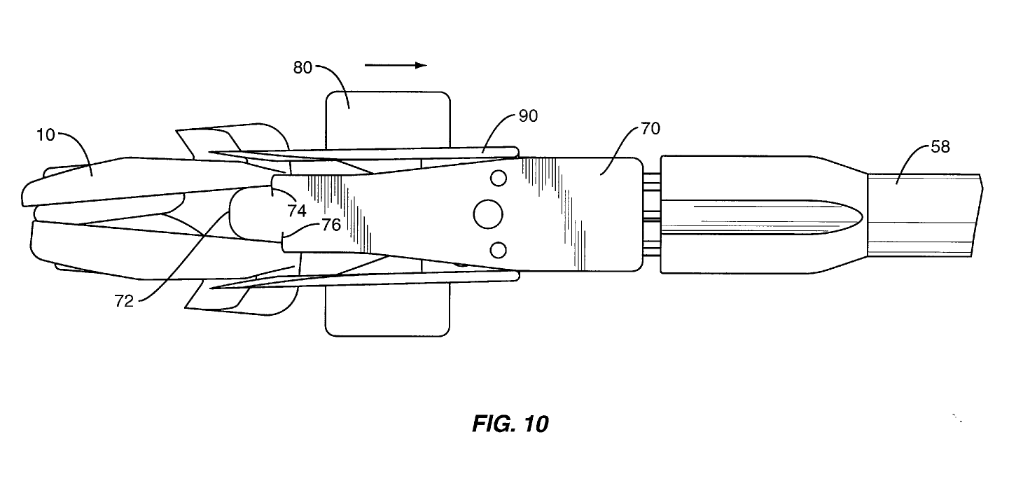

The slider 80 is moveably coupled to the driver tip portion 70 so as to slide distally-proximally between a forward clamping position (FIG. 8) and a rearward release position (FIG. 10). In the clamping position, the slider's engagement face 88 engages the corresponding clamping arm 90, such as by bearing on the cross-piece 98 thereof. In this position, the interface between the two is advantageously a rearward portion of the sloped engagement face 88, a portion that is relatively closer to the driver tip portion 70. By moving the slider 80 to the release position, a different position on the slope of the engagement face 88 engages the clamping arm 90, one that is relatively more proximal and therefore vertically farther from the driver tip portion 70. This allows the clamping arm 90, under the influence of bias spring 124, to pivot outward away from the driver tip portion 70. By pivoting outward, the clamping arm griping area 92 is brought out of engaging contact with the insert 10, thereby releasing the insert 10. The slider is advantageously biased forward to the clamping position, such as by bias element 126.

The tool may optionally include distraction blades 110 that are pivotally mounted to a suitable flange 38 on handle 30, outboard of the driver shaft assembly 40. The distraction blades 110 are generally plate-like structures with a proximal mounting end 112 and a distal forward portion 114. A slot 116 extends along most of the length of the distraction blade 110, and is advantageously open toward the forward portion 114. For most of its length, slot 116 is sized to receive slider 80, but is smaller than retention flanges 84, Further, the slot 116 may be widened toward the mounting end 112 so as to be able to accept retention flanges 84. The forward portion 116 may advantageously include a suitable projection 118 to help prevent over-insertion. For additional information regarding the distraction blades, attention is directed to U.S. patent application Ser. No. 10/764,621, filed 26 Jan. 2004, which is incorporated herein by reference in its entirety.

Before use, the driver shaft assembly 40 is retracted with respect to handle 30, with threads 56 engaging threads 36. The user may pivot one or both of the distraction blades 110 out of the way, due to the extra width of slots 116 near their mounting end 112. The user then pulls the slider 80 proximally in order to “open” the clamping arms 90. The insert 10 is placed so that a portion thereof is disposed between the gripping areas 92 of the clamping arms 90 and the driver distal tip 70, advantageously by abutting a portion of the insert 10 against the abutment faces 74,76. The slider 80 is then released, and biasing spring 126 pushes slider 80 forward, closing the clamping arms 90 to thereby clamp the insert 10 between the clamping arms 90 and the driver distal tip 70. It should be noted that with some embodiments of the tool 20, the insert 10 is advantageously held in a forwardly tapering configuration due to the relative offset of the abutment faces 74,76. The distraction blades 110 are then returned to their normal position. The tool 20 is then “loaded” and ready for use.

During the surgical procedure, the tool 20 is used to position the insert 10 in the desired location. Due the connection between the insert 10 and the tool 20, the combination may be handled as a unit, greatly facilitating the initial insertion process. When the distraction blades 110 are suitably located, the surgeon turns the proximal end 44 of the driver shaft assembly 40, such as by applying a rotation force to flanges 46. This rotation, due to the interaction of threads 36,56, causes driver shaft assembly 40 to advance relative to handle 30. As the distal tip 70 of driver shaft assembly 40 advances, slider 80 slides along slots 116 in distraction blades 110, with driver distal tip 70 and insert 10 disposed between the distraction blades 110, thereby helping to keep the forward portion of driver shaft assembly 40 properly aligned. In some embodiments, advancement of the driver tip 70 may cause the distraction blades 110 to spread apart, so as to aid in providing the proper space for the insert 10. As the driver shaft assembly 40 advances, threads 36,56 should, at some point, near the end of their mutual engagement. By this point in the travel of the driver shaft assembly 40, the insert 10 is advantageously at least partially inserted into the desired location. For example, the forwardmost portion of insert 10 may be disposed between the relevant vertebrae, with the rearmost portion of the insert 10 being still outside the vertebrae. This point advantageously coincides with the point where the slider 80 is just leaving the forward portion 114 of distraction blades 110 through the open end of slot 116. The insert 10 is still clamped at this point.

With additional turning of the driver proximal end 44, the bridging section 60 of driver shaft 40 becomes aligned with threads 36, thereby longitudinally decoupling the driver shaft 40 from the handle 30. The surgeon is then free to drive the insert 10 into position by impacting the proximal end 44 of drive shaft 40. For example, the surgeon may strike the impact insert 48 of the driver shaft 40 with a suitable impact device, such as a mallet. It should be noted that because threads 36 do not engage the bridging section 60, the driver shaft 40 is free to slide relative to the handle 30 in response to the impact, but that handle 30 may remain stationary. Thus, the surgeon may comfortably hold the handle 30 to keep the tool 20 in proper alignment while striking the proximal end 44, thus maintaining better control. The impact force on the proximal end 44 is transmitted to the insert 10 by the driver shaft assembly 40, driving the insert 10 into position. The impact force transmission is aided by the abutment faces 74,76, of the distal tip 70 pushing against the proximal faces of the insert's superior plate 12 and inferior plate 16. The insert 10 is advantageously still gripped by clamping arms 90 during the initial portion of this impacting. In order to help prevent the clamping arms 90 from being disengaged from the insert 10, the insert may advantageously include shallow recesses in the area of contact with the clamping arms 90, so that the outboard surfaces of the clamping arms 90 are slightly recessed relative to the insert's surfaces.

As the insert 10 is driven forward, the slider abutment faces 86 abut against the nearby bone (e.g., the posterior face of the vertebrae), impeding further forward motion of the slider 80. Therefore, as the driver shaft 40 continues to move forward, such as in response to the impact loading, the slider 80 is forced to slide rearward relative to the driver tip 70. This movement causes engagement point between the clamping arms 90 and the slider 80 to move outward along the slope of the engagement face 88. This action allows clamping arms 90 to move outward under bias from spring 124, thereby changing the relative angle α between the clamping arms 90 and axis 42, from a more inward angle to a less inward angle (e.g., level or outward). When the slider 80 has been forced rearward to the release position, the gripping areas 92 of the clamping arms 90 are moved outward sufficiently to disengage from the insert 10, thereby automatically releasing the insert 10 as discussed above. The tool 20 may then be used to further drive the insert 10 into position, or may be removed, as is appropriate.

It should be noted that in some embodiments the rearward relative travel of the slider 80 is advantageously positively stopped at a point that sets a maximum amount of “counter-sinking” of the insert. For example, the tool 20 may be used to drive the insert forward until the slider 80 hits the rear wall of slot 78, or until the slider 80 fully compresses spring 126, which acts to stop the rearward travel of the slider 80 relative to the driver tip 70. When the slider 80 cannot travel further rearward, this means that the driver tip 70 cannot travel farther forward (relative to the slider 80), which in turn means that the insert cannot be driven farther forward. Thus, this positive stop action prevents over-insertion of the tool.

The surgical tool 20 may be made from any suitable materials, such as stainless steel, titianium, titanium alloys, or any other suitable materials known in the art.

In some embodiments, the tool 20 may be used in conjunction with an insert 10 that includes an outwardly extending flange or “fin” 14. The tool 20 for such an insert 10 should have a corresponding notch in the forward portion of the clamping arms 90 so as to partially receive the insert's flange 14. Further, the slider 80 should be configured to extend farther outward than the insert's flange 14 so as to enable the slider's abutment faces 86 to abut the nearby bone appropriately.

The discussion above has assumed that the tool 20 includes two clamping arms 90; however, this is not required in all embodiments. For example, an embodiment of tool 20 could include only a single clamping arm 90, if desired, that operates as described above. For such an embodiment, the slider 80 need not have two engagement faces 88, etc. Further, the discussion above has assumed that the clamping arm(s) 90 are pivotally mounted to the driver tip 70. However, other methods of attaching the clamping arms 90 may be used. For example, the clamping arms 90 may be made of a suitable resilient material (e.g., spring steel), and be affixed to the driver tip 70 at their proximal ends 100. For such an embodiment, the clamping arm(s) 90 would be deflected to allow their distal ends 92 to move inward toward and outward away from the driver tip 70. However, due to the expected gripping forces, a relatively rigid gripping arm 90 is believed to be advantageous, and pivoting mounting of such a gripping arm 90 is likewise believed to be advantageous.

The discussion above has assumed that the slider 80 maintains engagement with the clamping arms 90 in the release position, albeit at an outer portion of the engagement faces 88. However, this is not required, and the clamping arms 90 may, if desired, not be constrained by the slider 80 in the release position.

The various aspects of the surgical tool 20, such as the insert auto-release feature, the selectively longitudinally coupled/decoupled driver shaft assembly 40, and other aspects, may be found individually in various embodiments of the surgical tool 20, or in any combination. Further, it is contemplated that the surgical tool 20 may be used for installation of an insert 10 from a posterior, anterior, lateral, oblique, or any other surgical approach.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A surgical tool for manipulating a surgical insert, comprising:
a housing;
an elongate driver moveably coupled to said housing and forwardly extending therefrom, said driver having a distal tip portion;
a first forwardly extending arm coupled to said driver tip portion and having a first distal end portion biased away therefrom;
a slider slidably coupled to said driver tip portion and moveable between a forward clamping position and a rearward release position;
wherein moving said slider from said release position to said clamping position urges said distal end portion of said first arm toward said driver tip portion for gripping the surgical insert therebetween; and
wherein moving said slider from said clamping position to said release position allows said distal end portion of said first arm to move away from said driver tip portion for releasing the surgical insert.

2. The surgical tool of claim 1 wherein said first arm pivotably couples to said driver tip portion.

3. The surgical tool of claim 1:
further comprising a second forwardly extending arm coupled to said driver tip portion and disposed generally opposite said first arm relative to said driver tip portion; said second arm having a second distal end portion biased away from said driver tip portion;
wherein moving said slider from said release position to said clamping position urges said distal end portion of said second arm toward said driver tip portion for gripping the surgical insert therebetween; and
wherein moving said slider from said clamping position to said release position allows said distal end portion of said second arm to move away from said driver tip portion for releasing the surgical insert.

4. The surgical tool of claim 3 further comprising an elastic element biasing said first and second arms away from said driver tip portion.

5. The surgical tool of claim 1 wherein said slider is forwardly biased relative to said driver.

6. The surgical tool of claim 1 wherein said slider comprises an engagement surface that is at least partially sloped, wherein said engagement surface engages said first arm in said clamping position.

7. The surgical tool of claim 1 wherein said slider further comprises a forwardly facing abutment face disposed outwardly relative to said first arm when said slider is in said clamping position.

8. The surgical tool of claim 1 wherein said first arm comprises a slot, and wherein said slider extends through said slot in at least said clamped position.

9. The surgical tool of claim 1 wherein said driver is elongate in a first direction and wherein said driver tip portion comprises a first abutment surface disposed generally transverse to said first direction and spaced from a distal endface of said driver tip portion.

10. The surgical tool of claim 9 wherein said driver tip portion comprises a second abutment surface disposed generally transverse to said first direction and spaced from said distal endface and said first abutment surface.

11. The surgical tool of claim 10 wherein said first and second abutment surfaces are unequally spaced from said distal endface.

12. The surgical tool of claim 1 wherein said driver comprises:
a proximal drive-force-receiving end section disposed generally opposite said driver tip portion;
an intermediate drive section disposed between said force-receiving end section and said driver tip portion;
a bridging section disposed between said drive section and said force-receiving end section, said bridging section configured to allow said driver to freely longitudinally slide relative to said handle in response to a proximally directed longitudinal force applied at said force-receiving end section.

13. The surgical tool of claim 15 wherein said intermediate drive section comprises an external thread and said handle comprises an internal thread for mating with said external thread.

14. The surgical tool of claim 1:
wherein said first arm pivotably couples to said driver tip portion;
further comprising a second forwardly extending arm pivotally coupled to said driver tip portion and disposed generally opposite said first arm relative to said driver tip portion; said second arm having a second distal end portion biased away from said driver tip portion;
wherein moving said slider from said release position to said clamping position urges said distal end portion of said second arm toward said driver tip portion for gripping the surgical insert therebetween; wherein moving said slider from said clamping position to said release position allows said distal end portion of said second arm to move away from said driver tip portion for releasing the surgical insert;
further comprising an elastic element biasing said first and second arms away from said driver tip portion;
wherein said slider comprises first and second engagement surfaces that are at least partially sloped, wherein said first and second engagement surfaces engage said first and second arms, respectively, in said clamping position; wherein said slider is biased toward said clamping position;
wherein said first and second arm each comprise a slot, and wherein said slider extends through said slots in at least said clamped position; and
wherein said driver is elongate in a first direction, and wherein said driver tip portion comprises first and second abutment surfaces disposed generally transverse to said first direction and spaced from a distal endface of said driver tip portion, wherein said driver tip portion comprises a second abutment surface disposed generally transverse to said first direction and spaced from said distal endface and said first abutment surface; wherein said first and second abutment surfaces are unequally spaced from said distal endface.

15. The surgical tool of claim 14:
wherein said slider further comprises a forwardly facing abutment face disposed outwardly relative to said first arm when said slider is in said clamping position;
wherein said driver comprises:
a proximal drive-force-receiving end section disposed generally opposite said driver tip portion;
an intermediate drive section disposed between said force-receiving end section and said driver tip portion;
a bridging section disposed between said drive section and said force-receiving end section, said bridging section configured to allow said driver to freely longitudinally slide relative to said handle in response to a proximally directed longitudinal force applied at said force-receiving end section; and
wherein said intermediate drive section comprises an external thread and said handle comprises an internal thread for mating with said external thread.

16. A surgical tool for manipulating a surgical insert, comprising:
a housing having a bore therethrough with first threads;
an elongate driver moveably coupled to said housing and extending through said bore to forwardly extend from said housing;
wherein said driver comprises:
a distal tip portion for mating with the insert;
a proximal drive force-receiving end section disposed generally opposite said tip portion;
an intermediate threaded section disposed between said force-receiving end section and said tip portion and having second threads configured to engage said first threads;
a bridging section disposed between said intermediate section and said force-receiving end section; said bridging section smaller than said intermediate section;
wherein, when said first threads engage said second threads, said driver couples to said handle for longitudinal movement therewith;
wherein, when said first threads are aligned with said bridging section, said driver is longitudinally decoupled from, and slidable relative to, said handle.

17. The surgical tool of claim 16 further comprising a gripping means associated with said driver tip portion for releasably gripping the insert.

18. The surgical tool of claim 16 wherein said distal tip portion is rotatable relative to said intermediate threaded section.

19. The surgical tool of claim 16 wherein said proximal drive force-receiving end section comprises an impact block disposed generally coaxially with said intermediate threaded section.

20. The surgical tool of claim 16 wherein said driver is elongate in a first direction and wherein said driver tip portion comprises a first abutment surface disposed generally transverse to said first direction and spaced from a distal endface of said driver tip portion.

21. The surgical tool of claim 20 wherein said driver tip portion further comprises a second abutment surface disposed generally transverse to said first direction and spaced from said distal endface and said first abutment surface.

22. The surgical tool of claim 21 wherein said first and second abutment surfaces are unequally spaced from said distal endface.

23. A method of using a surgical tool for manipulating a surgical insert, comprising:
biasing a first moveable clamping arm outward away from a distal tip of the tool;
attaching the insert to the tool by clamping the insert between the first clamping arm and the distal tip of the tool by sliding a slider relative to the distal tip;
manipulating the insert at least partially into a desired position relative to a bony structure in a patient;
abutting the slider against a portion of the bony structure;
retracting the slider, by advancing the tool, to allow the first clamping arm to move away from the distal tip to at least partially release the insert.

24. The method of claim 23 further comprising biasing a second clamping arm outward away from the distal tip; wherein said attaching the insert to the tool further comprises clamping the insert between the second clamping arm and the distal tip of the tool.

25. The method of claim 24 wherein said retracting the slider further allows the second clamping arm to move away from the distal tip.

26. The method of claim 23 wherein said attaching the insert to the tool comprises abutting the insert against an abutment face of the distal tip; wherein the abutment face is proximal to a distal endface of the distal tip.

27. A method of using a surgical tool for manipulating a surgical insert, comprising:
attaching the insert to the tool;
rotating a driver shaft assembly relative to a handle to advance the insert relative to the handle;
thereafter, further advancing the insert, while still attached to the tool, with the driver shaft assembly longitudinally decoupled from the handle.

28. The method of claim 27 further comprising releasing the insert from the tool while said driver shaft assembly is longitudinally decoupled from said handle.

29. The method of claim 28 wherein said tool comprises a slider longitudinally moveably mounted to said driver shaft assembly, and further comprising preventing further forward advancement of the driver shaft assembly by stopping the rearward movement of the slider relative to the driver shaft assembly.

30. The method of claim 27 wherein said further advancing the insert comprises applying an impact force to said driver shaft assembly while said driver shaft assembly is longitudinally decoupled from the handle.
